(19) United States
(12) Patent Application Publication
(10) Pub. No.: US 2005/0070501 A1
(43) Pub. Date: Mar. 31, 2005

(54) Water dispersible film

(75) Inventors: Alexander Robert Neurath, New York, NY (US); Nathan Strick, Oceanside, NY (US); Yun-Yao Li, Fresh Meadows, NY (US)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: NEW YORK BLOOD CENTER, INC., New York (US)

(21) Appl. No.: 10/918,915

(22) Filed: Aug. 16, 2004

Related U.S. Application Data
(60) Provisional application No. 60/507,072, filed on Sep. 29, 2003.

(51) Int. Cl. A61K 31/716

(52) U.S. Cl. 514/57

(57) ABSTRACT

A soft, pliable cellulose acetate phthalate (CAP)—hydroxypropyl cellulose (HPC) composite film is provided which is generated by casting from organic solvent mixtures containing ethanol. The film rapidly reduces the infectivity of several sexually transmitted disease pathogens, including the human immunodeficiency virus (HIV-1), herpesvirus (HSV), non-viral sexually transmitted disease pathogens (such as Neisseria gonorrhoeae, Haemophilus ducreyi, Chlamydia trachomatis and Treponema pallidum) and bacteria associated with bacterial vaginosis (BV). The film is converted into a gel/cream and thus does not have to be removed following application and use. In addition to being a topical microbicide, the film can be employed for the mucosal delivery of pharmaceuticals other than cellulose acetate phthalate.

### CROSS-REFERENCE TO RELATED APPLICATION

The present application claims the benefit of priority under 35 USC 102(e) for U.S. Provisional application Ser. No. 60/507,072 filed Sep. 29, 2003.

### GOVERNMENT RIGHTS

This invention was made with United States government support under Grant PO1 HD41761 from the National Institute of Health (“NIH”). The United States government may have certain rights in this invention.

### BACKGROUND OF THE INVENTION

1. Field of the Invention

The present invention concerns a water dispersible film that can be used as a microbicide to prevent the sexual transmission of the human immunodeficiency virus, herpesviruses, and non-viral sexually transmitted disease pathogens and as a drug delivery system. The present invention is also directed to a method of making such film. More particularly, the present invention relates to a water dispersible microbicidal containing hydroxypropyl cellulose (“HPC”) and cellulose acetate phthalate (“CAP”) film.

2. Background of the Invention

Polymers used in the past as pharmaceutical excipients and in drug delivery, are increasingly being considered for specific therapeutic and prophylactic applications (Liao J., Ottenbrite R. M., “Biological effects of polymeric drugs”, In Controlled Drug Delivery. Edited by Park K. Washington, DC: American Chemical Society; 1997, 455-467; Uglea C. V., Panaitescu L., “Synthetic polyanionic macromolecules with antiviral and antitumoral activity”, Current Trends in Polymer Science, 1997, 2: 241-251; Chiellini E., Sunamoto J., Migliaresi C., Ottenbrite R. M., Cohn D., (Ed), Proceedings of the Third International Symposium on Frontiers in Biomedical Polymers including Polymer Therapeutics: From Laboratory to Clinical Practice: 23-27, May 1999; Shiga. Dordrecht: Kluwer Academic/Plenum Publishers: 2001; Duncan R., “The dawning era of polymer therapeutics”, Nat Rev Drug Discov., 2003, 2: 347-360; Kabanov A. V., Okano T., “Challenges in polymer therapeutics: State of the art and prospects of polymer drugs”, In Polymer drugs in the clinical stage, Edited by Maeda H., Kabanov A., Kataoka K., Okano T., New York: Kluwer Academic/Plenum Publishers; 2003, 1-27). Such polymers may appear to be promising for topical applications such as microbicides to prevent infection by sexually transmitted disease (STD) pathogens, including the human immunodeficiency virus (HIV-1) (Stone A., “Microbicides: A new approach to preventing HIV and other sexually transmitted infections”, Nat. Rev. Drug Discov, 2002, 1: 977-985).

One of these promising polymeric microbicides is cellulose acetate phthalate (CAP). (Neurath A. R., Strick N., Li Y-Y., Lin K., Jiang S., “Design of a ‘microbicide’ for prevention of sexually transmitted diseases using ‘inactive’ pharmaceutical excipients”, 1999, 27: 11-21; Gyotoku T., Aurelian L., Neurath A. R., “Cellulose acetate phthalate (CAP): an ‘inactive’ pharmaceutical excipient with antiviral activity in the mouse model of genital herpesvirus infection”, Antiviral Chem. Chemother., 1999, 10: 327-332; Manson K. H., Wyand M. S., Miller C., Neurath A. R., “The effect of a cellulose acetate phthalate topical cream on vaginal transmission of simian immunodeficiency virus in rhesus monkeys”, Antimicrob. Agents Chemother., 2000, 44: 3199-3202; Neurath A. R., Li Y-Y., Mandeville R, Richard L, “In vitro activity of a cellulose acetate phthalate topical cream against organisms associated with bacterial vaginosis”, J Antimicrob Chemother., 2000, 45: 713-714; Kawamura T., Cohen S. S., Borris D. L., Aquilino E. A., Glushakova S., Margolis L. B., Orenstein J. M., Offord R., Neurath A., Blauvelt A., “Candidate microbicides block HIV-1 infection of human immature Langerhans cells within epithelial tissue explants”, J Exp Med., 2000, 192: 1491-1500; Neurath A. R., Strick N., Li Y-Y., Debnath A. K., “Cellulose acetate phthalate, a common pharmaceutical excipient, inactivates HIV-1 and blocks the coreceptor binding site on the virus envelope glycoprotein gp120”, BMC Infect. Dis., 2001, 1: 17; Neurath A. R., Strick N., Jiang S., Li Y-Y., Debnath A. K., “Anti-HIV-1 activity of cellulose acetate phthalate: Synergy with soluble CD4 and induction of ‘dead-end’ gp41 six-helix bundles”, BMC Infect. Dis., 2002, 2: 6; and Neurath A. R., Strick N., Li Y-Y., “Anti-HIV-1 activity of anionic polymers: A comparative study of candidate microbicides”, BMC Infect. Dis., 2002, 2: 27).

CAP has been used for enteric film coating of tablets and capsules (Goskonda S. R., Lee J. C., “Cellulose Acetate Phthalate”, In Handbook of Pharmaceutical Excipients, Edited by Kibbe A. H. Washington, D.C./London, U.K.: American Pharmaceutical Association/Pharmaceutical Press; 2000:99-101) and thus has a well-established safety record for human use. CAP is not soluble in water pH<≈5.8. For this reason, it must be used in a micronized form for both tablet coating from water dispersions, and as a topical microbicide. Micronization is accomplished by pseudolatex emulsion processes (Banker G. S., “Pharmaceutical coating composition, and preparation and dosages so coated”, U.S. Pat. No. 4,330,338, 1982; McGinley E. J., Tuason D. C., “Enteric coating for pharmaceutical dosage forms”, U.S. Pat. No. 4,518,433, 1985; McGinley E. J., “Enteric coating for pharmaceutical dosage forms”, European Patent EP 0 111 103, 1989; Wu S. H. W., Greene C. J., Sharma M. K., “Water-dispersible polymeric compositions”, U.S. Pat. No. 4,960,814; 1990; Wu S. H. W., Greene C. J., Sharma M. K., “Water-dispersible polymeric compositions”, U.S. Pat. No. 5,025,004; 1991; Sakellariou P., Rowe R. C., “Phase separation and morphology in ethylcellulose/cellulose acetate phthalate blends”, J. Applied Polymer Science, 1991, 43, 845-855; Ibrahim H., Bindschaedler C., Doelker E., Buri P., Gurny R., “Aqueous nanodispersions prepared by a salting-out process”, Int. J. Pharm., 1992, 87, 239-246; Quintanar-Guerrero D., Allemann E., Fessi H., Doelker E., “Pseudolatex preparation using a novel emulsion-diffusion process involving direct displacement of partially water-miscible solvents by distillation”, Int. J. Pharm., 1999, 188, 155-164; Yuan J., Wu S. H. W., “Process for production of polymeric powders” U.S. Pat. No. 6,541,542; 2003). The entire content of each of the above-described following U.S. patents is hereby incorporated by reference herein: U.S. Pat. No. 4,330,338; U.S. Pat. No. 4,518,433; U.S. Pat. No. 4,960,814; U.S. Pat. No. 5,025,004; and U.S. Pat. No. 6,541,542.

A micronized form of CAP available commercially under the trade name “Aquateric” (FMC Corporation, Philadelphia, Pa., USA) (containing approximately 63 to 70 weight % CAP, poloxamers and acetylated monoglycerides) in appropriate gel formulations was shown to inactivate HIV-1 and several other STD pathogens in vitro and in animal models (Neurath et al., Biologicals, (1999), 27, 11-21; Gyoku et al., Antiviral Chem. Chemother., (1999), 10, 327-33; Manson et al., Antimicrob. Agents Chemother. 2000, 44, 3199-3202; Neurath et al., BMC Infect. Dis., (2002), 2, 7). Micronized CAP was shown to be the only candidate microbicide having the capacity to remove HIV-1 rapidly by adsorption from physiological fluids and render the virus noninfectious.

CAP or hydroxypropylmethylcellulose phthalate (HPMEP) has been employed to decrease the frequency of transmission of human immunodeficiency virus or herpesvirus infections (U.S. Pat. No. 5,985,313 and U.S. Pat. No. 6,165,493, both to Neurath et al.); and to treat or prevent bacterial vaginosis (U.S. Pat. No. 6,462,030 to Neurath et al.).

Microbicidal gels with or without contraceptive activity have disadvantages. They need applicators for topical delivery which adds to cost and generating disposal problems (which is an environmental concern). These drawbacks can be overcome by unit dose biodegradable devices dispersible in water having the following properties: (1) the microbicidal activity is a built-in property of the device, i.e., the active ingredient is an integral structural component of the device; (2) the device absorbs physiological fluids and then disintegrates; (3) infectious agents bind to the resulting structures and become rapidly inactivated; and (4) lastly, the device is converted into a soft gel which does not have to be removed. One such biodegradable microbicidal vaginal barrier device is a sponge prepared by freeze-drying a foam generated from a water suspension of Aquateric in a solution of bioadhesive partially substituted ethers of cellulose (e.g., hydroxypropyl methylcellulose, methylcellulose, hydroxyethyl cellulose and hydroxypropyl cellulose (HPC) (U.S. Pat. No. 6,572,875 to Neurath and Strick)). Another biodegradable microbicidal vaginal barrier device which comprises CAP or hydropropylmethylcellulose phthalate (HPMCP) and a pectin is described in U.S. Pat. No. 6,596,297 to Neurath and Strick.

Alternatively, the sponges can be prepared by freeze-drying a microemulsion (Kietzke T., Neher D., Landfester K., Montenegro R., Guntner R., Scherf U., “Novel approaches to polymer blends based on polymer nanoparticles”, Nat. Mater, 2003, 2: 408-412) of CAP in ethyl acetate mixed with a water solution of one of the cellulose ethers (U.S. Pat. No. 6,572,875). These sponges contained 34 to 40 weight % of the active ingredient, CAP. The advantages of the unit dose sponges are extenuated by the relatively high cost of freeze-drying. This would limit their use as a microbicide in developing countries. Therefore, alternative approaches had to be explored.

Water soluble or dispersible films have been used for drug delivery onto mucosal surfaces (Heusser J, Martin M., “Pharmaceutical, vaginal applicable preparation and a process for its preparation”, U.S. Pat. No. 5,380,529; 1995; Meyers M, “Use of edible film to prolong chewing gum shelf life”, U.S. Pat. No. 5,409,715; 1995; Staab R., “Dissolvable device for contraception or delivery of medication”, U.S. Pat. No. 5,529,782; 1996; Thombre A. G., Wigman L. S., “Rapidly disintegrating and fast-dissolving solid dosage form”, U.S. Pat. No. 6,497,899; 2002).

### SUMMARY OF THE INVENTION

It is an object of the present invention to furnish a water dispersible microbicidal cellulose phthalate film.

It is another object of the present invention to provide a water dispersible film that can be used as a drug delivery system.

It is a further object of the present invention to provide a method for producing such water dispersible film.

It is moreover another object of the present invention to treat bacterial vaginosis or prevent human immunodeficiency virus, herpesvirus infections and other sexually transmitted diseases.

The present invention serves to avoid the aforementioned difficulties with microbicidal gels by replacing such gels/creams with unit dose biodegradeable devices which are dispersible in physiological fluids such as seminal fluid or vaginal secretions.

The present invention provides a mucoadhesive film which is converted in the presence of water into a smooth cream containing micronized CAP. The present invention thus concerns a water dispersible film comprising cellulose acetate phthalate, hydroxypropyl cellulose and glycerol, the film when dried contains 35 to 45 weight % of the cellulose acetate phthalate, 35 to 45 weight % of the hydroxypropyl cellulose and 10 to 30 weight % of the glycerol, said film after sufficient contact with water or a physiological fluid, is converted into a gel or cream containing micronized cellulose acetate phthalate.

The present invention further concerns a drug delivery system. Thus, the present invention is directed to a composition comprising (i) a composite comprising cellulose acetate phthalate, hydroxypropyl cellulose and glycerol, the composite when dried in an organic solvent contains 35 to 45 weight % of the cellulose acetate phthalate, 35 to 45 weight % of the hydroxypropyl cellulose and 10 to 30 weight % of the glycerol, and (ii) a pharmaceutically effective amount of a pharmaceutical that is capable of being dissolved in said organic solvent.

The present invention also relates to a method of preventing human immunodeficiency virus, herpesviruses, and non-viral sexually transmitted disease infections in a human in need thereof by applying to a mucous membrane of such human the film of the present invention.

The present invention further concerns a method of treating bacterial vaginosis by vaginally administering to a woman the film of the present invention.

The present invention is also directed to a method of producing such film by combining CAP with hydroxypropyl cellulose (HPC) and casting from organic solvent mixtures containing ethanol. Accordingly, the present invention provides a method of producing a water dispersible film comprising dissolving cellulose acetate phthalate, hydroxypropyl cellulose and glycerol in an organic solvent mixture comprising ethanol and another organic solvent selected from the group consisting of acetone, ethyl acetate and glacial acetic acid, wherein the cellulose acetate phthalate is in an amount of 1.75 weight % or more, the hydroxypropyl cellulose is in an amount of 1.75 weight % or more, the glycerol is in an amount of 0.75 weight % or more, with the remainder being the organic solvent mixture, as long as the dried film has the same composition as the dried film as described above (35 to 45 weight % of CAP, 35 to 45 weight % of HPC and 10 to 30 weight % of glycerol). The film is cast from this mixture using appropriate film casting and drying equipment.

### BRIEF DESCRIPTION OF THE DRAWINGS

For the purpose of illustrating the invention, drawings are provided. It is to be understood, however, that the present invention is not limited to the precise arrangements and instrumentalities depicted in the drawings.

FIGS. 1A to 1F concern the morphology of a selected composite film hereinafter referred to as “H” of cellulose acetate phthalate (CAP) and hydroxypropyl cellulose (HPC) and particles after film dispersion in water.

FIG. 1A is a scanning electron micrograph (“SEM”) of film H (side “A” exposed to air during drying).

FIG. 1B is a 3-dimensional (3-D) interactive display of side “A” of film H.

FIG. 1C is a 3-D interactive display of film H (side “B” in contact with the casting surface during drying).

In FIG. 1B and FIG. 1C, the bar at the bottom corresponds to an elevation scale.

FIG. 1D is a graph showing the kinetics of conversion of shredded film H into a cream as measured by an increase of viscosity, wherein the circles represent H₂O and the squares represent seminal fluid.

FIG. 1E is a SEM of CAP particles from a cream formed from the film.

The scale bar in the right-hand corner below the drawings for each of FIG. 1A and FIG. 1E is 1μ.

FIG. 1F is a bar graph showing the size distribution of the particles.

FIGS. 2A to 2D are graphs which show the inactivation of HIV-1 IIIB, HIV-1 BaL and herpesviruses HSV-1 and HSV-2 by graded quantities of the film H. Serial dilutions of the respective control and film treated (5 minutes at 37° C.) viruses were added to cells and virus replication was monitored by measuring β-galactosidase (β-gal) activity. In FIGS. 2A to 2D, the circles represent “untreated”; the squares represent a 56 mg/ml film; the diamonds represent a 28 mg/ml film; the triangles pointing upward represent a 14 mg/ml film; and the triangles pointing downward represent a 7 mg/ml film.

FIG. 2A is a graph for HIV-1 IIIB.

FIG. 2B is a graph for HIV-1 BaL.

FIG. 2C is a graph for HSV-1.

FIG. 2D is a graph for HSV-2.

FIG. 3 is a bar graph showing the inactivation by film H of selected non-viral STD pathogens and bacteria associated with bacterial vaginosis (BV). The STD pathogens (Neisseria gonorrhoeae, Haemophilus ducreyi and Chlamydia trachomatis) and bacteria associated with bacterial vaginosis (BV) (Gardnerella vaginalis, Mycoplasma capricolum and Mycoplasma hominis) were treated with graded quantities of the film H for 5 minutes to 37° C. The abscissa of FIG. 3 indicates that film dosages for Chlamydia trachomatis were different from those used for the other bacteria.

FIG. 3 depicts four groups of six bars. The six bars from left to right represent, respectively, Neisseria gonorrhoeae, Haemophilus ducreyi, Chlamydia trachomatis, Gardnerella vaginalis, Mycoplasma capricolum and Mycoplasma hominis.

FIG. 4 is a graph showing the kinetics of conversion of a film into a gel in water. The HPC used in this film has a viscosity grade of 75 to 150 cps.

### DETAILED DESCRIPTION OF THE INVENTION

In one embodiment of the present invention, a soft, flexible composite film is provided in which the active ingredient, CAP, is an integral structural component. The film, when dried, includes hydroxypropyl cellulose (HPC) and glycerol. Preferably the hydroxypropyl cellulose component has a viscosity grade of 75 to 6,500 cps. A sufficient amount of glycerol is used to make the film soft.

The dried film contains 35 to 45 weight % CAP (preferably 38 to 42 weight % CAP), 35 to 45 weight % HPC (preferably 38 to 42 weight % HPC) and 10 to 30 weight % glycerol (preferably 16 to 24 weight % glycerol).

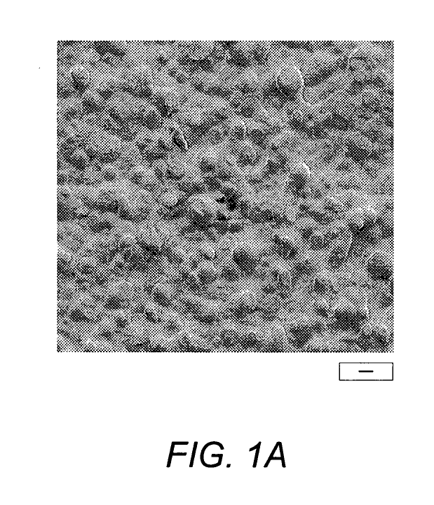

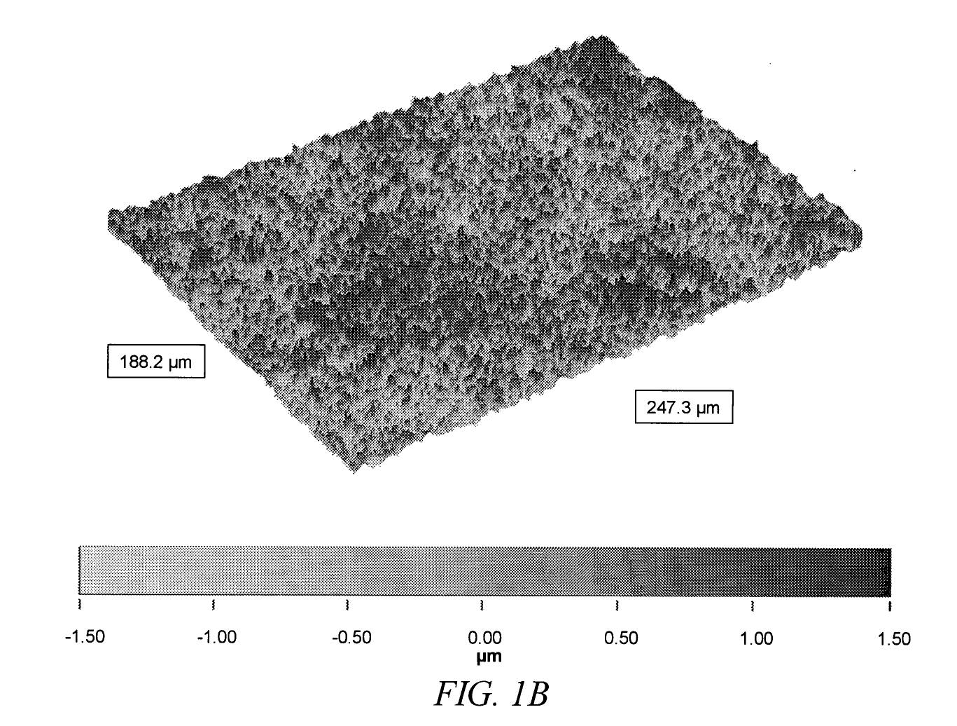

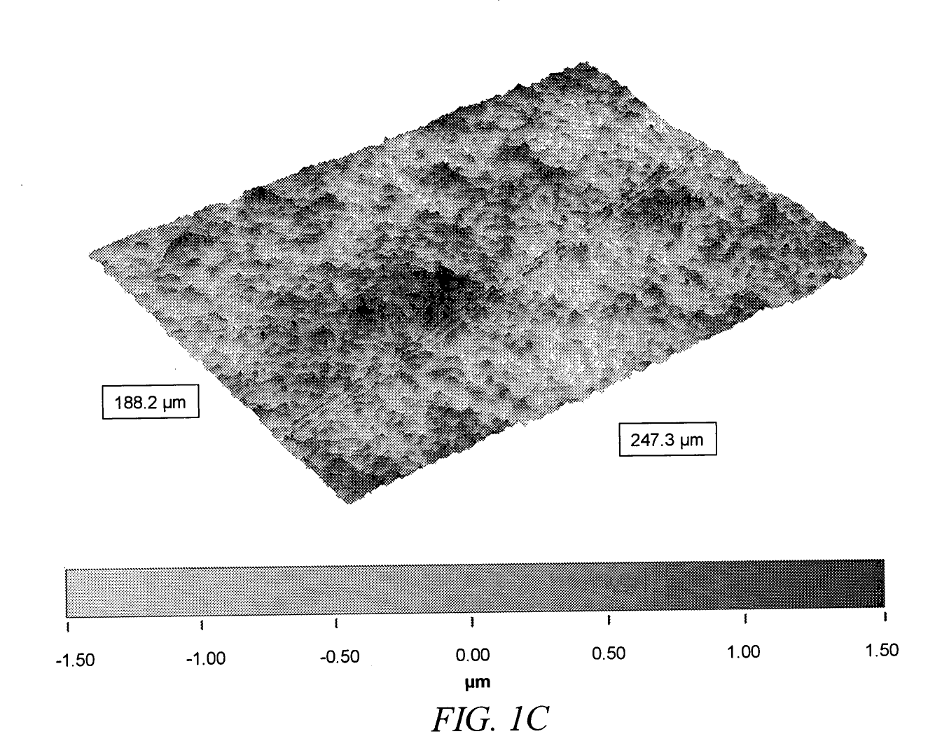

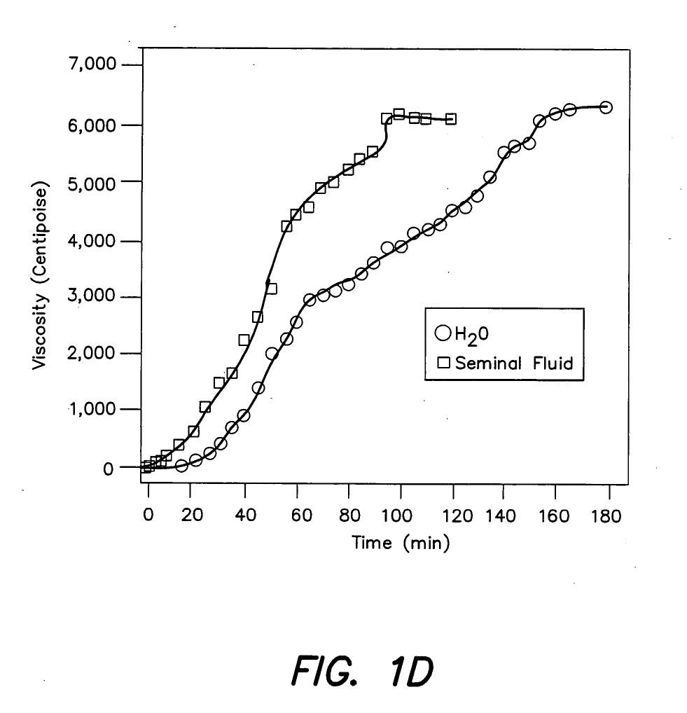

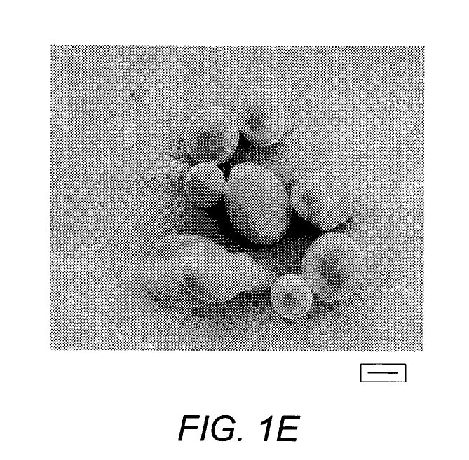

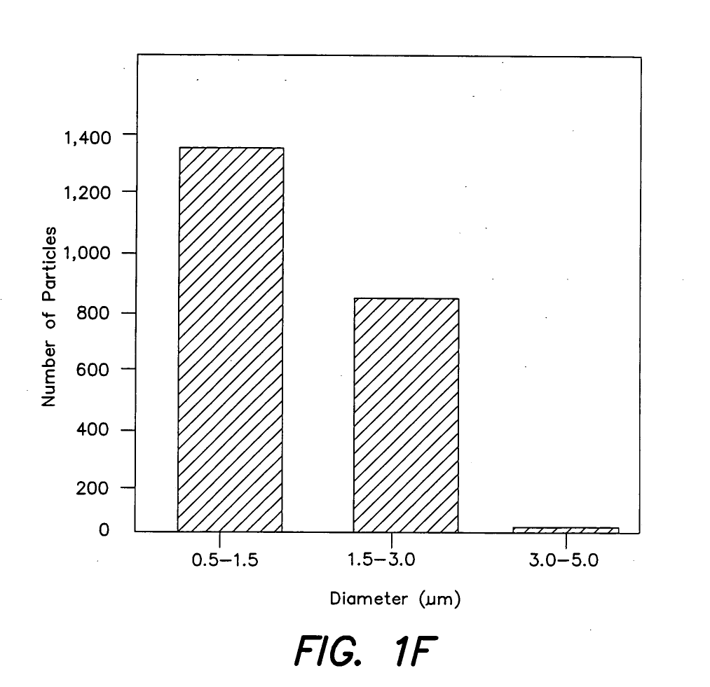

The film of the present invention absorbs water and disintegrates, leading to the formation of micronized CAP particles which were shown to adsorb HIV-1 (Neurath et al., BMC Infect. Dis., (2002), 2, 27) and inactivate STD pathogens. Thus, the CAP-HPC composite film of the present invention, after sufficient contact with water or a physiological fluid, is progressively converted into a gel/cream (FIG. 1D), thus obviating the need for delivery by an applicator. Similar gels were shown earlier (Neurath et al., Biologicals, (1999), 27, 11-21, Gyotoku et al., Antiviral Chem. Chemother., (1999), 10, 327-332; Neurath et al., BMC Infect. Dis., (2001), 1, 17; Neurath et al., BMC Infect. Dis., (2002), 2, 6; Neurath et al., BMC Infect. Dis., (2002), 2, 27) to rapidly inactivate HIV-1, HSV and other STD pathogens.

Upon contact with fluids containing STD pathogens, the film of the present invention inactivates viruses and/or bacteria rapidly, long before its conversion into a gel. Expected exposure to high sheer rates during physiological processes would result in more rapid disintegration and conversion of the film into a gel than shown in FIG. 1D. This indicates that the film will be efficacious under in vivo conditions.

Similarly to CAP based gels (Neurath et al., J. Antimicrob. Chemother., (2000), 45, 713-714), the CAP-HPC film of the present invention is active against several bacteria associated with BV, known to increase susceptibility to HIV-1 infection (Martin H. L., Jr., Richardson B. A., Nyange P., Lavreys L., Hillier S. L., Chohan B, Mandaliya K., Ndinya-Achola J. O., Bwayo J., Kreiss J. “Vaginal lactobacilli, microbial flora, and risk of human immunodeficiency virus type 1 and sexually transmited disease acquisition”, J. Infect. Dis., 1999, 180: 1863-1868). Thus inserted CAP-HPC films can be used for the treatment of BV.

The film of the present invention can be applied to a mucous membrane of a man or a woman for preventing human immunodeficiency virus (HIV-1), herpesvirus (HSV-1 or HSV-2), and non-viral sexually transmitted disease infections (such as Neisseria gonorrhoeae, Haemophilus ducreyi, Chlamydia trachomatis and Treponema pallidum) or treating bacterial vaginosis (BV). Thus the film can be applied to an internal body area such as the vagina, rectum, oral cavity, nasal passage, etc.

The film may contain additives such as preservatives, flavors, fragrances and/or coloring agents. These additives may be present in any desired concentration. The concentrations of these additives will depend upon the desired properties, the agent to be released, the potency, the desired dosage, dissolution times, etc.

In another embodiment of the present invention, the CAP-HPC composite film can be used for delivery to mucosal surfaces of pharmaceuticals other than CAP. The pharmaceutical should be a drug that can be dissolved in the organic solvent used to make the film, such as acetone. Such applications with respect to mucosal surfaces include oral and ophthalmic applications (Gates K. A., Grad H., Birek P., Lee P. I., “A new bioerodible polymer insert for the controlled release of metronidazole”, Pharm. Res., 1994, 11: 1605-1609; Baeyens V., Kaltsatos V., Boisrame B., Fathi M., Gurny R., “Evaluation of Soluble Bioadhesive Ophthalmic Drug Inserts (BODI) for prolonged release of gentamicin: lachrymal pharmacokinetics and ocular tolerance”, J. Ocul. Pharmacol. Ther., 1998, 14:263-272).

Non-limiting types of pharmaceuticals that can be delivered in this manner include antibiotics, anti-viral agents, fungicides, anaesthetics, anti-inflammatory agents, anti-itch agents, spermicides, analgesics and antiseptics.

Combined with other excipients, the shredded composite film of the present invention can be compressed into tablets which disintegrate instantaneously, providing an alternative microbicide and general drug delivery system.

The CAP-HPC composite can be dried from organic solvent mixtures containing ethanol (EtOH) (as described herein) in physical forms other than a film, e.g., granules, combined with tablet disintegrants (Mannogem or Pharmaburst [SPI Pharma, Grand Haven, Mich., USA]) and compressed into tablets. The tablets in contact with water disintegrate instantaneously and are subsequently converted into a smooth cream similar to that generated by the films (FIG. 1D). Such tablets extend the potential application of the CAP-HPC composite as a topical microbicide and drug delivery tool. In general, the described composite contributes to broadening the function of CAP from an enteric coating material to becoming a component of novel mucosal drug delivery systems with inherent anti-microbial properties.

The tablets can be formed with any drug powder. The drug powder does not necessarily have to be able to dissolve in an organic solvent. Suitable drugs which can be employed in this manner include, but are not limited to, the following: (1) anti-infectives, such as antibiotics, e.g., azithromycin, trovafloxacin and sulfonamides, antivirals, antifungals, e.g., fuconazole and voriconazole, antiprotozoan and antibacterials; (2) anti-inflammatories, such as hydrocortisone, oxaprozin, celecoxib, valdecoxib, dexamethasone, triamcinolone, and various prednisolone compounds; (3) estrogenic steroids, such as estrone; (4) progestational agents, such as progesterone; (5) prostaglandins; (6) coronary vasodialators and other drugs for treating coronary disorders; (7) antitussives; (8) antihistamines, e.g., cetirizine; (9) anesthetics, (10) anti-hypertensives, e.g., indormin, amlodipine and nifedipine; (11) analgesics, e.g., meptazinol and pentazocine; (12) tranquilizers, e.g., lorazepan, oxazepan and tempazepan; (13) contraceptives, e.g., ethnyl estradiol and norgestral; (14) psychotropics; (15) cough/cold remedies, including decongestants; (16) drugs for the treatment of Alzheimer's disease, such as donepezil; (17) drugs for the treatment of urinary incontinence, e.g., darifenacin; (18) drugs for the treatment of osteoporosis, e.g., droloxfene; (19) muscle relaxants, e.g., orphenadrine; (20) aldose reductase inhibitors, e.g., zopolrestat; (21) neuromucular drugs, e.g., pyridostigmine; (22) gonadal hormones; (23) corticosteroids, e.g., prednisolone; (24) HGM-CoA reductase inhibitors, e.g., atorvasatin; (25) drugs acting on the uterus, e.g., hyoscine butyl bromide; (26) anti-allergics, e.g., triprolidine; (27) drugs for relieving poisoning; (28) drugs for metabolic dysfunction, e.g., methysergide; (29) drugs for the treatment of male erectile dysfunction, e.g. sildenifil; (30) drugs for the treatment of diabetes, e.g., glipizide; (31) drugs for the treatment of migraine headache, e.g., eletriplan, sumatriptan; and (32) adrenergic antagonists, e.g., doxazosin. Other specific drugs that can be used include clotrimazole, miconazole, ticonazole, benzalkonium chloride, nystatin, benzocaine and nitroglycerine.

Combinations of the various drugs may be used as desired. Typically the range of the drug may be in the amount of 0.0001% to about 5% by weight. The drug may be in a variety of chemical forms, such as uncharged molecules, molecular complexes, or nonirritating, pharmacologically acceptable salts. Simple derivatives of such drugs, such as ethers, esters, amides, and the like, can also be used for desirable properties such as retention, release, and easy hydrolyzation by body pH, enzymes, etc. The amount of drug to be used varies depending upon the particular drug, the desired therapeutic or prophylactic effect, and required release times.

In a further embodiment of the present invention, a method is provided to produce the water dispersible films of the present invention. Such method involves dissolving CAP, hydroxypropyl cellulose (HPC) and glycerol in ethanol and another organic solvent such as acetone, and transferring (such as by pouring) the resultant mixture into a container such as a dish or plate, such as a Teflon® coated or aluminum plate, or solid polymeric material, from which the dried film can easily be removed. Preferably a solvent mixture is employed containing almost equal to 50 to almost equal to 65 weight % ethanol. Then the solvent or solvent mixture is evaporated by drying.

For preparing the film of the present invention, it is preferable to employ 0.2 to 3 weight % CAP; 2 to 5 weight % of HPC; 0.8 to 1.2 weight % glycerol, with the remainder being the organic solvent which includes ethanol and another organic solvent such as ethyl acetate, glacial acetic acid and acetone. It is preferred that the other organic solvent be acetone.

Unlike the vacuum drying of porous frozen foam (resulting in sponges), the drying of cast films does not result in sufficient removal of water. The residual moisture would render the films unstable during storage above room temperature due to the slow hydrolysis of CAP (Goskonda et al., Handbook of Pharmaceutical Excipients, (2000), 99-101; Gates et al., Pharm. Res., (1994), 11, 1605-1609; Karlsson A., Singh S. K., “Thermal and mechanical characterization of cellulose acetate phthalate films for pharmaceutical tablet coating: Effect of humidity during measurements”, Drug Dev. Ind. Pharm., 1998, 24: 827-834).

Problems resulting from residual moisture in CAP films cast from water suspensions could theoretically be overcome by preparing the films from organic solvents. However, this appeared counterintuitive since CAP films cast from organic solvents are water resistant (Goskonda et al., Handbook of Pharmaceutical Excipients, (2000), 99-101), and start dissolving only at pH>≈5.8. Furthermore, none of the mucoadhesive cellulose ethers used together with CAP/Aquateric for production of sponges (U.S. Pat. No. 6,572,875) has been reported to be soluble in organic solvents which dissolve CAP (Goskonda et al., Handbook of Pharmaceutical Excipients, (2000), 99-101), except for HPC which is soluble in methylene chloride (R. J. Hawood, “Hydropropyl Cellulose”, Handbook of Pharmaceutical Excipients, edited by A. H. Kibbe, Washington, D.C., London, U.K., American Pharmaceutical Association, Pharmaceutical Press, (2000), 244-248). HPC is also one of the best bioadhesive polymers among cellulose ethers (K. R. Tambweker, V. K. Gujan, R. Kandarapu, L. J. D. Zaneveld, S. Garg, “Effect of Different Bioadhesive Polymers on Performance Characteristics of Vaginal Tablets”, Microbicides 2002 Conference Abstract, 15 (2002).

Composite CAP (for example, 40 weight %)—HPC (for example, 40 weight %)—glycerol (for example, 20 weight %) films can be cast from one of the following anhydrous organic solvents: ethyl acetate; glacial acetic acid; methylene chloride; and acetone/EtOH 9:1 (v/v). It was found that the resulting films were hard, brittle and did not disperse in water. Surprisingly, the addition of EtOH (final concentrations of 50 to 65 weight %) to the casting solvents ethyl acetate, CH₃COOH and acetone, respectively, resulted in films with dramatically altered properties. The films were soft, flexible, and dispersed in water, resulting ultimately in smooth creams. The properties of a selected film (designated “H”) containing 40 weight % CAP, 40 weight % HPC and 20 weight % glycerol cast from acetone/EtOH 4:6 are described herein.

### EXAMPLES

The present invention will now be described with reference to the following non-limiting examples.

### Example 1: Preparation and Physical Properties of CAP-HPC Film

CAP, HPC (150-400 cps, NF, Spectrum, New Brunswick, N.J., USA), HPC (4,000-6,500 cps, NF, Spectrum) and glycerol were dissolved in acetone-ethanol (EtOH) 4:6 at final concentrations of 2, 1, 1, and 1% (w/w), respectively. The viscous liquids were poured into Teflon® coated steel or aluminum foil dishes (0.425 g/cm²) which were subsequently maintained for 16 hours at 40° C. followed by 1 hour in a vacuum oven at 50° C. to dry the films.

To measure the kinetics of film conversion into a cream, the film was shredded into ≈1 mm² pieces in a Guardian Cross-Cut Shredder (Quartet GBC, Skokie, Ill., USA) and added at 75 mg/ml to either water or human seminal fluid (New England Immunology Associates, Cambridge, Mass., USA). The viscosity was measured in a DV-3 P R digital viscometer (Anton Paar GmbH, Graz, Austria) using a TR-8 spindle at speeds decreasing from 200 to 2 r.p.m.

Imaging of cast films was performed with a JEOL 6500 Field Emission scanning electron microscope (SEM) (JEOL USA, Inc., Peabody, Mass., USA) at a magnification of 5,000×. Scanning white light interferometric microscopy (“SWLIM”) was performed on both sides of the film at a magnification of 25×. The scanning electron micrographs of film H (thickness≧100μ) revealed a particle-accumulated layer on one side (side A; exposed to air during drying) of the film (FIG. 1A) while the other side was smooth (results not shown). This is also shown in the 3-dimensional interactive display of both sides of the film (FIG. 1B, FIG. 1C).

CAP particles obtained after complete dispersion of the film were pelleted by centrifugation at 10,000×g for 5 minutes, washed with water to remove excess HPC, and freeze dried. The particles were dispersed in water and measured by automated scanning electron microscopy using a JEOL 6400 scanning electron microscope coupled with a NORAN Voyager system (NORAN Instruments, Inc., Middleton, Wis., USA). Imaging of the particles on a carbon substrate was performed using the JEOL 6500 electron microscope.

Exposure of the film to water resulted in disintegration and formation of smaller particles ultimately convertible into a cream. Mixing of pieces of film in water at low speed resulted in the generation of a smooth cream as indicated by a gradual increase of viscosity (FIG. 1D), which was more rapid when the film was suspended in seminal fluid. SEM revealed particles of micronized CAP in the resulting cream (FIG. 1E). The particles had a size between 0.5 to 3× (FIG. 1F).

### Example 2: Measurements of Infectivity of HIV-1 and Herpesviruses (HSV)

To measure HIV-1 infectivity, virus was precipitated from tissue culture media containing 10% fetal bovine serum with polyethylene glycol 8000 (final concentration 10 mg/ml). The pellet containing virus was dissolved in 225 μl aliquots of 0.14 M NaCl, 0.01 M Tris(hydroxymethyl)aminomethane, pH 7.2 (TS). The aliquots were pre-warmed to 37° C. and precut pieces of a film “H” were added. After 5 minutes at 37° C., 1.225 ml of tissue culture medium were added and the mixtures were centrifuged for 1 hour at 14,000 r.p.m. in an Eppendorf 54156 microfuge (Brinkmann Instruments, Inc., Westbury, N.Y., USA) to pellet the virus. The virus was redissolved, serially diluted twofold (2× to 2,048×), and the dilutions tested for infectivity using HeLa-CD4-LTR-β-gal and MAGI-CCR5 cells obtained from the AIDS Reagent and Reference Reagent Program (Rockville, Md., USA) for HIV-1 IIIB and HIV-1 BaL, respectively.

Virus replication was quantitated by measuring β-galactosidase (β-gal) activity in cell lysates as described in Neurath et al., BMC Infect. Dis., (2002) 2, 27. In a parallel series of experiments, residual film H was removed by centrifugation at 2,000 r.p.m. for 5 minutes from the film-virus mixtures before pelleting the virus at 14,000 r.p.m. The infectivities of control and film H treated HSV-1 and HSV-2, respectively, were measured under similar conditions as described for HIV-1 (Neurath et al., Biologicals, (1999), 27, 11-21). HSV-1 was in the form of a recombinant virus, vgCL5, in which the expression of β-galactosidase (β-gal) is under the control of the late gene C regulatory region. Vero cells were used for infection which was monitored by measuring β-gal activity. ELVIS HSV cells (Diagnostic Hybrids, Inc., Athens, Ohio, USA), containing a LacZ gene placed behind an inducible HSV promoter, were used for infection by HSV-2. Infection was determined by measuring β-gal.

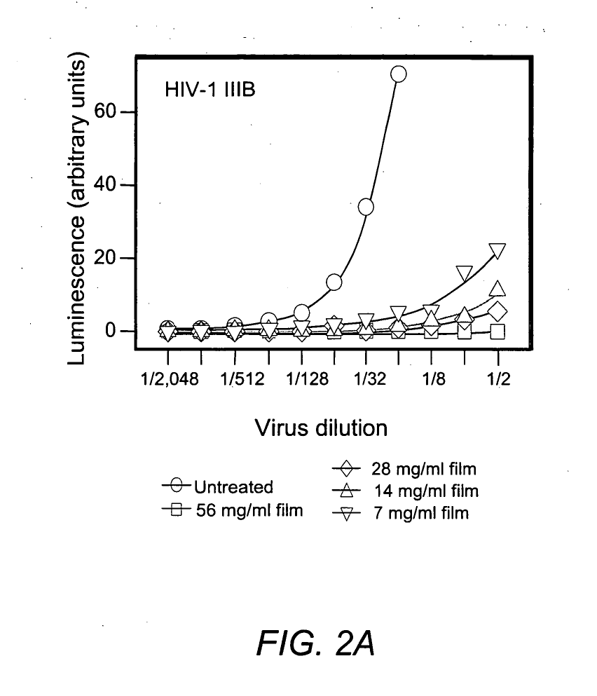

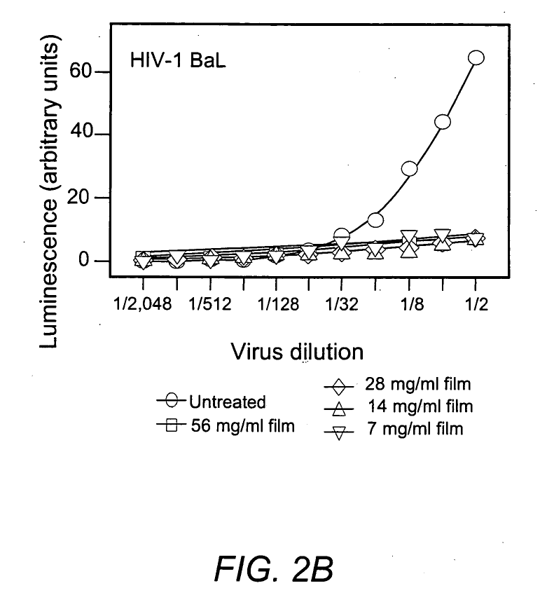

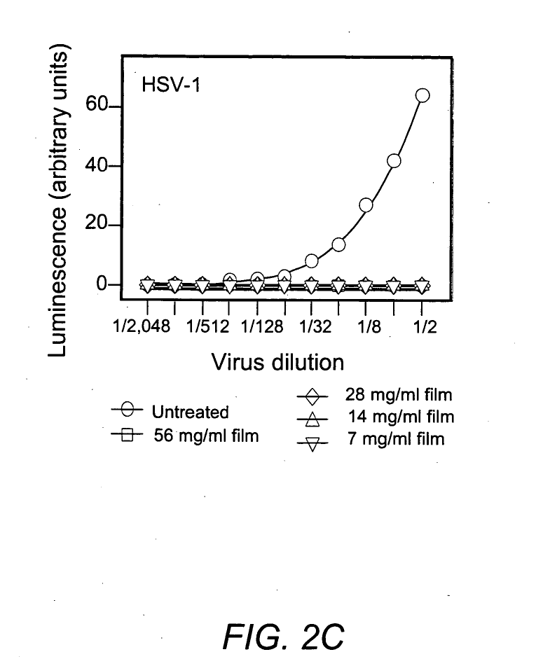

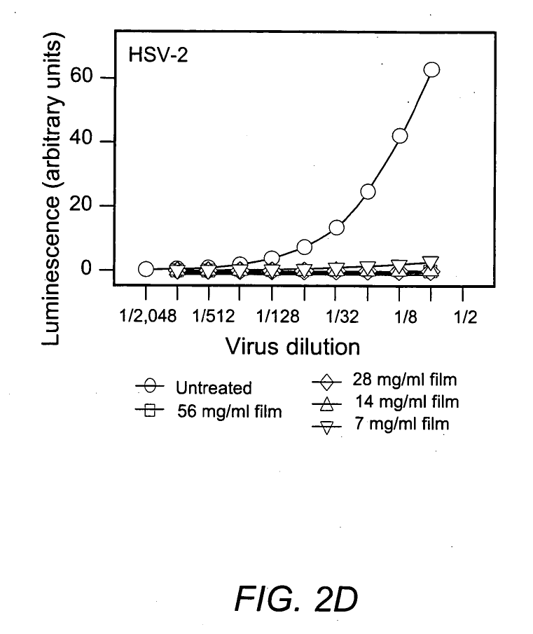

Micronized CAP (Aquateric) has been shown to inactivate within a few minutes the infectivity of HIV-1, HSV and several non-viral STD pathogens (Neurath et al., Biologicals, (1999), 27, 11-21; Neurath et al., BMC Infect. Dis., (2002)). It was of interest to determine whether film H, long before it completely disintegrates in the presence of water, and is converted into a cream, has similar effects. At the highest dose of film (56 mg/ml)≧99% inactivation of HIV-1, HSV-1 and HSV-2 was observed within 5 minutes at 37° C. (FIG. 2A to FIG. 2D). Both HIV-1 IIIB and BaL, viruses utilizing distinct cellular coreceptors, CXCR4 and CCR5, respectively (Neurath et al., BMC Infect. Dis., (2001), 1, 17), were inactivated. As the film dose was reduced, the extent of virus inactivation diminished and was 89±4, 82±9, 99.7±0.1, and 95±2% for HIV-1 IIIB, HIV-1 BaL, HSV-1 and HSV-2, respectively, at a dose of 7 mg/ml. The residual infectivity in all cases was recovered in supernatants after removing film and particles released from it by centrifugation, suggesting that only virus not adsorbed to the film material escaped inactivation. This was confirmed in separate experiments (data not shown). For comparison, the suggested unit dose of film as a microbicide is ≈1,000 mg

### Example 3: Inactivation of Non-Viral STD Pathogens and Bacteria Associated with Bacterial Vaginosis (BV)

The bacterial strains and the corresponding growth media were obtained from the American Type Culture Collection (ATCC, Manassas, Va., USA) and were the same as described in Neurath et al., Biologicals, (1999), 27, 1, 11-21 and Neurath et al., J. Antimicrob. Chemother., (2000), 45, 713-714). The Mycoplasma capricolum that was used was ATCC # 23205. Graded quantities of film H (0 to 150 mg/ml) were added to suspensions of the respective bacteria (8×10⁸ to 1×10⁹/ml in TS) pre-warmed to 37° C. After 5 minutes at 37° C., the suspensions were diluted 10-fold in the appropriate growth medium, centrifuged to pellet the bacteria which were then resuspended in the original volume of growth medium. Serial 10-fold dilutions in the appropriate growth media were made, and after incubation at 37° C. (30° C. for Haemophilus ducreyi) for 20 hours to 5 days, depending on the bacterial strain, turbidity was measured at 600 nm. Serial twofold dilutions (100 μl) of control and film H treated Chlamydia trachomatis were added to 9×10⁴ McCoy cells plated into wells of 96-well microtiter plates. After 48 hours, the cells were fixed and stained with fluorescein isothiocyanate labeled monoclonal antibodies to Chlamydia (Diagnostic Hybrids) and the fluorescent inclusion bodies were counted following the procedures provided by the manufacturer.

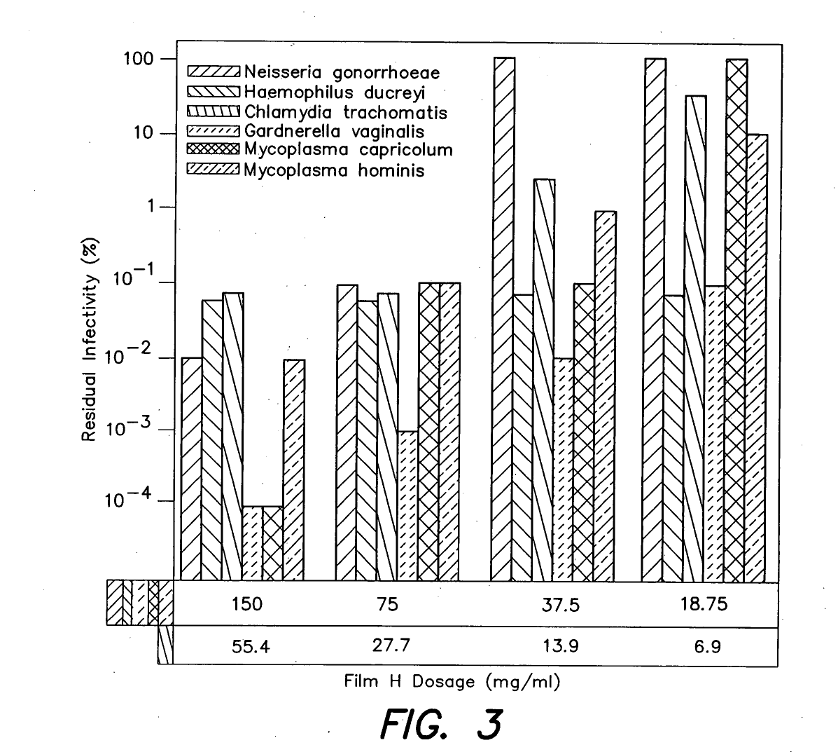

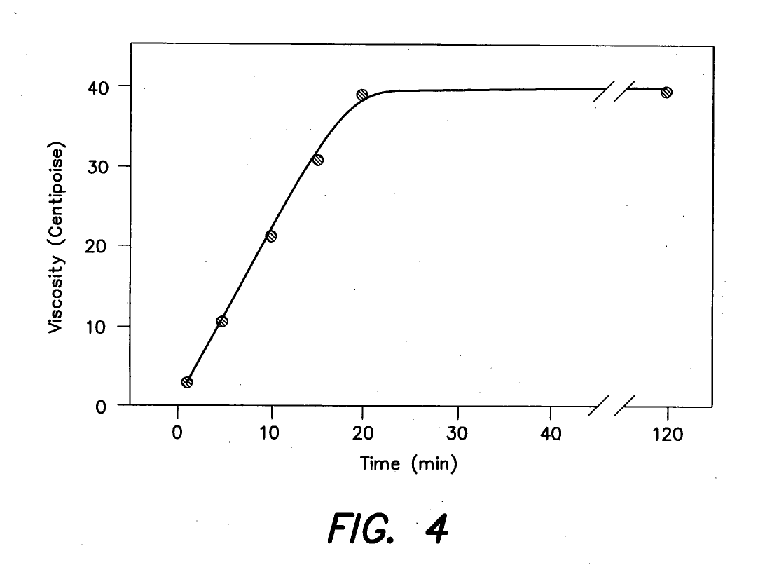

Film H also inactivated several non-viral STD pathogens (FIG. 3). This effect can be attributed to the low pH provided by CAP (Neurath et al., Biologicals, (1999), 27, 11-21; Neurath et al., J. Antimicrob. Chemother., (2000), 45, 713-714), unlike the anti-HIV-1 and anti-HSV-1/-2 effects occurring at both acidic and neutral pH (Neurath et al., BMC Infect. Dis., (2002), 2, 6; Neurath et al., BMC Infect. Dis., (2002), 2, 27). Thus, the films, before complete conversion into a cream, reduced the infectivity of non-viral STD pathogens>1,000-fold at doses≧75 mg/ml (≧27.7 mg/ml for Chlamydia trachomatis).

It will be appreciated that the instant specification is set forth by way of illustration and not limitation, and that various modifications and changes may be made without departing from the spirit and scope of the present invention.

1. A water dispersible film comprising cellulose acetate phthalate, hydroxypropyl cellulose and glycerol, the film when dried contains 35 to 45 weight % of the cellulose acetate phthalate, 35 to 45 weight % of the hydroxypropyl cellulose and 10 to 30 weight % of the glycerol, said film after sufficient contact with water or a physiological fluid, is converted into a gel or cream containing micronized cellulose acetate phthalate.

2. The film according to claim 1, wherein the cellulose acetate phthalate is in an amount of 38 to 42 weight %.

3. The film according to claim 1, wherein the hydroxypropyl cellulose is in an amount of 38 to 42 weight %.

4. The film according to claim 1, wherein the glycerol is in an amount of 16 to 24 weight %.

5. The film according to claim 1, wherein the hydroxypropyl cellulose has a viscosity grade of 75 to 6,500 cps.

6. A method for preventing HIV-infection comprising administering to a mucous membrane of a human a pharmaceutically effective anti-HIV-1 amount of the film according to claim 1.

7. A method for preventing herpesvirus-1 infection comprising administering to a mucous membrane of a human a pharmaceutically effective anti-herpesvirus-1 amount of the film according to claim 1.

8. A method for preventing herpesvirus-2 infection comprising administering to a mucous membrane of a human a pharmaceutically effective anti-herpesvirus-2 amount of the film according to claim 1.

9. A method for treating bacterial vaginosis comprising vaginally administering to a woman a pharmaceutically effective anti-bacterial vaginosis amount of the film according to claim 1.

10. A method for preventing a non-viral sexually transmitted disease infection comprising administering to a mucous membrane of a human in need thereof a pharmaceutically effective anti-non-viral sexually transmitted disease amount of the film according to claim 1.

11. The method according to claim 10, wherein the non-viral sexually transmitted disease infection is Chlamydia trachomatis infection.

12. The method according to claim 10, wherein the non-viral sexually transmitted disease infection is Neisseria gonorrhoeae infection.

13. The method according to claim 10, wherein the non-viral sexually transmitted disease infection is Haemophilus ducreyi infection.

14. The method according to claim 10, wherein the non-viral sexually transmitted disease infection is Treponema pallidum infection.

15. A composition comprising (i) a composite comprising cellulose acetate phthalate, hydroxypropyl cellulose and glycerol, the composite when dried in an organic solvent contains 35 to 45 weight % of the cellulose acetate phthalate, 35 to 45 weight % of the hydroxypropyl cellulose and 10 to 30 weight % of the glycerol, and (ii) a pharmaceutically effective amount of a pharmaceutical that is capable of being dissolved in said organic solvent.

16. The composition according to claim 15, wherein the pharmaceutical is selected from the group consisting of an antibiotic, an anti-viral agent, a fungicide, an anaesthetic, an anti-inflammatory agent, a spermicide, an analgesic, an antiseptic, a steroid, a progestational agent, a coronary vasodialator, an antitussive, an antihistamine, an anti-hypertensive, a tranquilizer, a contraceptive, a psychotropic, a decongestant, a muscle relaxant, an aldose reductase inhibitor, a neuromuscular drug, a gonadal hormone, a corticosteroid, a HGM-CoA reductase inhibitor and an adrenergic antagonist.

17. The composition according to claim 15, wherein the pharmaceutical is contained in an amount of 0.0001 to 5% by weight.

18. A method of producing a water dispersible film comprising preparing a mixture by dissolving cellulose acetate phthalate, hydroxypropyl cellulose and glycerol in an organic solvent mixture comprising ethanol and another organic solvent selected from the group consisting of acetone, ethyl acetate and glacial acetic acid, wherein the cellulose acetate phthalate is in an amount of 1.75 weight % or more, the hydroxypropyl cellulose is in an amount of 1.75 weight % or more and the glycerol is in an amount of 0.75 weight % or more, with the remainder being the organic solvent mixture, and casting the mixture into equipment for drying and forming a film, wherein the resultant dried film contains 35 to 45 weight % of the cellulose acetate phthalate, 35 to 45 weight % of the hydroxypropyl cellulose and 10 to 30 weight % of the glycerol.

19. The method according to claim 18, wherein the hydroxypropyl cellulose has a viscosity grade of 75 to 6,500 cps.

20. The method according to claim 18, wherein said organic solvent mixture is ethanol mixed with acetone.

21. The method according to claim 18, wherein said organic solvent mixture is ethanol mixed with ethyl acetate.

22. The method according to claim 18, wherein said organic solvent mixture is ethanol mixed with glacial acetic acid.
